(19) United States
(12) Patent Application Publication
(10) Pub. No.: US 2009/0149427 A1
(43) Pub. Date: Jun. 11, 2009

(54) PHARMACEUTICALLY ACCEPTABLE SALTS AND HYDRATES OF RISEDRONIC ACID

(75) Inventors: Kreso Mihaljevic, Zagreb (HR); Blazenko Bajic, Zagreb (HR); Drazen Cavuzic, Zagreb (HR); Ernest Mestrovic, Zagreb (HR); Miroslav Zegarac, Zagreb (HR); Nada Kosutic Hulita, Zagreb (HR)

Correspondence Address: FOLEY & LARDNER LLP, 150 EAST GILMAN STREET, P.O. BOX 1497, MADISON, WI, 53701-1497, US

(73) Assignee: Pliva Hrvatska d.o.o.

(21) Appl. No.: 12/088,545

(22) PCT Filed: Sep. 4, 2006
(86) PCT No.: PCT/GB2006/003268

§ 371 Date: Sep. 2, 2008

(30) Foreign Application Priority Data
Sep. 30, 2005 (GB) 0519891.6

(51) Int. Cl. A61K 31/675; C07F 9/28; A61P 35/00

(52) U.S. Cl. 514/89; 546/22

(57) ABSTRACT

The present invention is concerned with new hydrated forms of risedronate salts, processes of preparing the new hydrated forms, pharmaceutical compositions containing the same, therapeutic uses thereof and methods of treatment employing the same. In particular, the present invention provides tri-sodium risedronate dihydrate.

The present invention is concerned with new hydrated forms of risedronate salts, processes of preparing the new hydrated forms, pharmaceutical compositions containing the same, therapeutic uses thereof and methods of treatment employing the same.

Different hydrates of a drug substance can have different chemical and physical properties, including melting point, chemical reactivity, apparent solubility, dissolution rate, optical and mechanical properties, vapor pressure and density. These properties can have a direct effect on the ability to process and/or manufacture a drug substance and a drug product, as well as on drug product stability, dissolution and bioavailability. Thus, different hydrates can affect the quality, safety and efficacy of a drug product.

There are a number of methods that can be used to characterize different hydrates of a drug substance, such as single crystal X-ray diffraction and also X-ray powder diffraction. Other methods, including microscopy, thermal analysis (e.g., differential scanning calorimetry, thermal gravimetric analysis, and hot-stage microscopy) and spectroscopy (e.g., infrared [IR], Raman, solid-state nuclear magnetic resonance [ssNMR]) are also helpful to further characterize different hydrate forms.

Risedronic acid is the international non-proprietary name of [1-hydroxy-2-(3-pyridinyl)ethylidene]bisphosphonic acid. Risedronic acid has the following structural formula

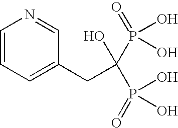

A particularly preferred salt of risedronic acid is risedronate sodium.

Bisphosphonic acids, such as risedronic acid, and pharmaceutically acceptable salts thereof, in particular risedronate sodium as referred to above, have been employed in the treatment of diseases of bone and calcium metabolism. Such diseases include osteoporosis, hyperparathyroidism, hypercalcemia of malignancy, ostolytic bone metastases, myosistis ossifcans progressiva, calcinoisis universalis, arthritis, neuritis, bursitis, tendonitis and other inflammatory conditions.

Bisphosphonic acids, and pharmaceutically acceptable salts thereof, tend to inhibit the resorption of bone tissue, which is beneficial to patients suffering from excessive bone loss. However, in spite of certain analogies in biological activity, all bisphosphonates do not exhibit the same degree of biological activity. Some bisphosphonates have serious drawbacks with respect to the degree of toxicity in animals and the tolerability or negative side effects in humans. The salt and hydrate forms of bisphosphonates alter both their solubility and their bioavailability.

Processes of preparing risedronic acid, and salts thereof, are known in the art, and some examples thereof are as follows.

EP 1243592B describes a process of preparing risedronic acid by reacting 3-pyridylacetic acid with phosphorous acid and phosphorous trichloride in a solvent. In the case where the solvent is chlorobenzene, the reaction is carried out at a temperature in the range of 85-100° C. In the case where the solvent is fluorobenzene, the reaction is carried out at the reflux temperature of the reaction medium. Isolation of the risedronic acid involves separation thereof from the reaction mixture by treatment with alkali metal or ammonium hydroxide, bicarbonate or carbonate and subsequent treatment of the resulting alkali metal or ammonium risedronic acid salt with a strong mineral acid.

EP 1252170B describes a process for selectively producing risedronate sodium hemipentahydrate or monohydrate comprising the steps of (a) providing an aqueous solution of risedronate sodium, (b) heating the aqueous solution to a temperature from about 45° C. to about 75° C., (c) adding a solvent to the aqueous solution, characterised in that the solvent is selected from the group consisting of alcohols, esters, ethers, ketones, amides and nitriles, and (d) optionally cooling the aqueous solution.

EP 04949844B also discloses a process of preparing bisphosphonic acids, but not risedronic acid. Bisphosphonic acids, in particular alendronic acid, of the following general formula are prepared according to the process of EP 0494844B

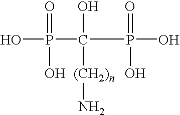

where n is 2 to 8. The process comprises melting a mixture of the corresponding aminocarboxylic acid and phosphorous acid in the absence of an organic solvent, adding dropwise phosphorous trihalide, adding to the reaction mixture a hydrolyzing agent selected between water and a strong non-oxidizing acid and recovering the diphosphonic acid thus produced. The process is described as being characterised in that the molar ratio between the aminocarboxylic acid, phosphorous acid and phosphorous trihalide in the reaction mixture is 1:3:2 and 1:20:6.

WO 03/086355 describes polymorph forms B, B1, BB, C, D, E, F, G and H of risedronate sodium and processes of preparing these various polymorphs.

WO 04/037252 discloses crystalline hydrated forms of sodium risedronate, which contain from 6.4 up to 22 weight % of sodium based on the anhydrous substance, and in the case where the sodium content is lower than 7.5 weight %, then 15 to 23 weight % of crystalline water is present, or in the case where the sodium content is higher than 7.5 weight %, then 4.5 to 18 weight % of crystalline water is present. Specifically, there is disclosed (i) the pentahydrate of the monosodium salt, which contains from 5.5 to 7.5 weight % of sodium and 20 to 23 weight % of crystalline water, (ii) the trihydrate of the trisodium salt, which contains from 19 to 21 weight % of sodium and 12 to 14 weight % of crystalline water and (iii) the monohydrate of the disodium salt, which contains from 13 to 15 weight % of sodium and 4.5 to 6.5 weight % of crystalline water.

WO 05/066190 discloses the following salts of risedronic acid, namely disodium risedronate, monopotassium risedronate, dipotassium risedronate, monoammonium risedronate, diammonium risedronate, hemipiperazine risedronate, ethanolamine risedronate and morpholinoethanolamine risedronate and hydrates thereof. Specifically, the following anhydrates and hydrates are disclosed, namely disodium risedronate anhydrate, disodium risedronate tetrahydrate, monopotassium risedronate dihydrate, dipotassium risedronate anhydrate, monoammonium risedronate monohydrate, monoammonium risedronate dihydrate, diammonium risedronate anhydrate, hemipiperazine risedronate anhydrate, ethanolamine risedronate anhydrate and morpholinoethanolamine risedronate anhydrate.

Acta Crystallographica, Section C: Crystal Structure Communications, 2003, Vol, C59(2), m33-m36 & Chemical Abstracts, abstract no 138:376654, describes three hydrates of risedronate which were obtained by varying the pH of a solution containing the compound. Specifically, the following risedronate hydrates were prepared—risedronate monohydrate, risedronate dihydrate and risedronate 2.5 hydrate.

The above discussed salts of risedronic acid, in particular the sodium salt of risedronic acid, known in the art to date have, however, been seen to suffer from stability and formulation problems in view of their hygroscopic nature. It is well recognized in the pharmaceutical field that hygroscopic materials (that is a material that readily absorbs water, usually from the atmosphere) are generally unstable and this instability can lead to problems in terms of shelf life and also formulation techniques. In many instances where such hygroscopic materials are employed in pharmaceutical formulations, specific steps need to be taken to protect the integrity of the hygroscopic materials.

There is a need, therefore, to provide a salt of risedronic acid in a form which is less hygroscopic than known salts of risedronic acid and which would, therefore, alleviate many of the instability and formulation problems associated with risedronic acid salts to date.

We have now, therefore, developed a new hydrated risedronate salt, which is distinguished from the disclosure of the prior art by the characteristics thereof as hereinafter described and which is particularly advantageous for use in pharmaceutical formulation in view of the non-hygroscopic properties thereof. More specifically, there is now provided by the present invention, a pharmaceutically acceptable tri-(alkali metal) salt of risedronic acid, which is present as the dihydrate form.

Pharmaceutically acceptable alkali metal salts include sodium and potassium salts. Specifically, there is provided by the present invention tri-sodium risedronate dihydrate.

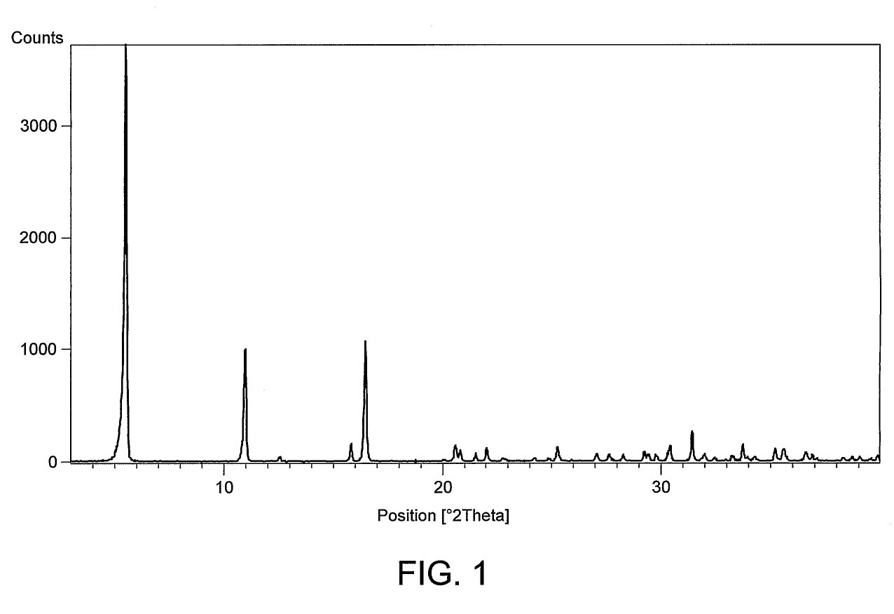

Tri-sodium risedronate dihydrate as provided by the present invention can be further characterised as having an X-ray powder diffraction pattern, or substantially the same X-ray powder diffraction pattern, as shown in FIG. 1. For the X-ray powder diffraction measurement, the instrument used was a Philips PANalytical X'PertPRO powder diffractometer and samples of tri-sodium risedronate dihydrate were prepared by powdering in a mortar and pestle, followed by direct application into an original circular sample holder (16 mm diameter), manually pressed with the Phillips' original sample preparation kit and closed with the Phillips' original bottom plate. Further operational details of the Philips PANalytical X'PertPRO powder diffractometer are shown in following Table 1.

TABLE 1

| | |
|---|---|
| Instrument | Philips X'Pert PRO |
| Operation voltage of generator | 45 kV, 40 mA |
| X-Ray tube | PW3373/10; Cu anode LFF |
| Focus | Line |
| Scan angle range (2Θ) | 3-40° |
| Scan mode | Continuous absolute scan |
| Step size (2Θ) | 0.016° |
| Time per step | 100 seconds |
| X-ray radiation filter | Nickel |
| X-ray radiation | CuKα |
| Primary soller slit | 0.04 rad |
| Primary mask | 10 mm |
| Secondary soller slit | 0.04 rad |
| Detector | X'Celerator |
| Control program | X'Pert Data Collector |

Tri-sodium risedronate dihydrate according to the present invention is further characterised as having characteristic peaks (2θ): 5.41±0.2°, 11.0±0.2° and 16.5±0.2°. Tri-sodium risedronate dihydrate according to the present invention is still further characterised by the following other typical peaks (2θ): 15.8±0.2°, 20.6±0.2°, 20.8±0.2°, 22.0±0.2°, 25.3±0.2°, 30.4±0.2°, 31.4±0.2° and 33.7±0.2°.

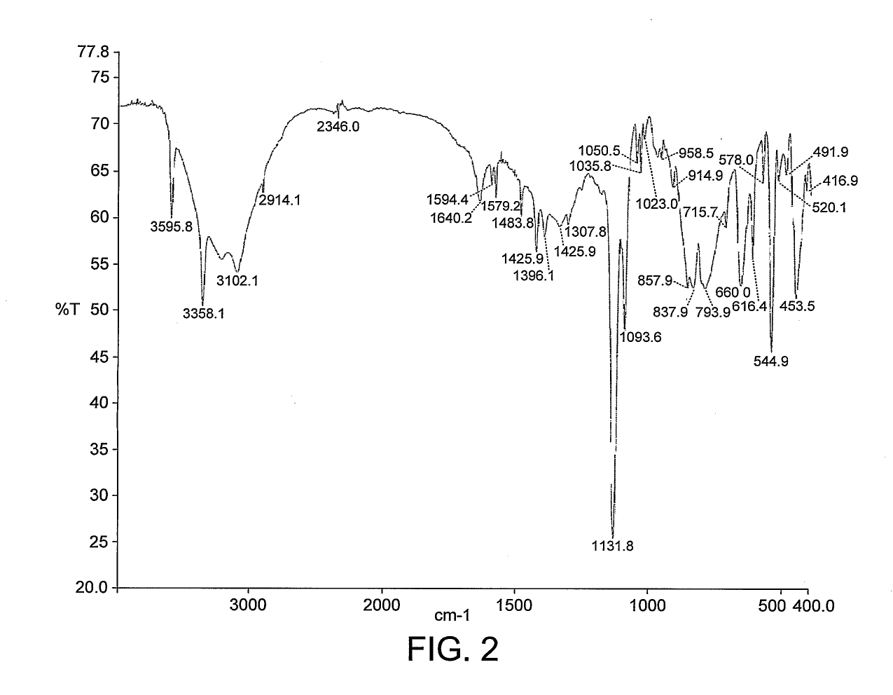

Tri-sodium risedronate dihydrate according to the present invention is further characterised as having an IR pattern, or substantially the same IR pattern, as shown in FIG. 2. More particularly, tri-sodium risedronate dihydrate has characteristic IR absorbance at about 3596±4, 3358±4, 3102±4, 1640±4, 1594±4, 15794, 1426±4, 1132±4, 1094±4, 958±4 and 545±4 cm⁻¹.

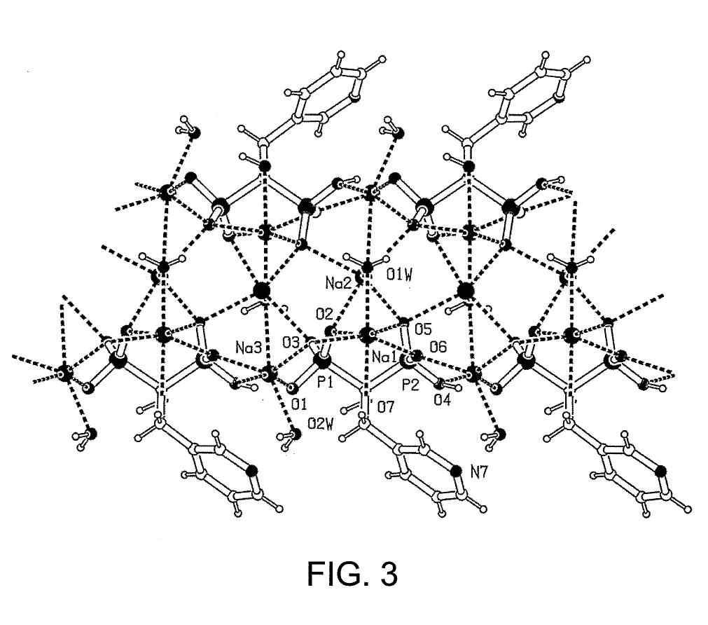

A single crystal of tri-sodium risedronate dihydrate was also prepared and single crystal X-ray diffraction data was collected using a Bruker Nonius FR591/Kappa CCD diffractometer with CuKα radiation giving the crystallographic data shown hereinafter. Specifically, the crystalline structure of tri-sodium risedronate dihydrate according to the present invention is shown in FIG. 3 and this is further characterized by an orthorombic space group P 2₁2₁2₁ and by displaying unit cell parameters comprising crystal axis lengths of a=5.70(2)Å, b=7.25(2)Å, c=32.28(4)Å. The crystalline structure of tri-sodium risedronate dihydrate is further characterized by the following properties:

TABLE 2
Crystallographic data:

| | |
|---|---|
| Chemical formula | C₇H₁₂N₁Na₃O₉P₂ |
| Empirical formula weight | 385.09 |
| Temperature | 293(2) K |
| Crystal system, space group | Orthorombic, P 2₁2₁2₁ |
| Unit cell dimension | a = 5.70(2) Å |
| | b = 7.25(2) Å |
| | c = 32.28(4) Å |
| Volume | 1333 (2) Å³ |
| Z | 4 |
| Calculated density | 1.92 (1) g/cm⁻³ |

TABLE 3
Atomic coordinates and equivalent isotropic displacement parameters
for tri-sodium risedronate dihydrate. (U(eq) is defined as one
third of the trace of the orthogonalized Uij tensor.)

| | x | y | z | U(eq) |
|---|---|---|---|---|
| Na(1) | 0.108 | −0.115 | 0.806 | 0.023 |
| Na(2) | 0.632 | −0.095 | 0.742 | 0.031 |
| Na(3) | 0.835 | −0.633 | 0.849 | 0.033 |
| P(1) | 0.594 | 0.106 | 0.834 | 0.016 |
| P(2) | 0.579 | −0.316 | 0.833 | 0.017 |
| O(1) | 0.684 | 0.253 | 0.865 | 0.025 |
| O(2) | 0.786 | 0.066 | 0.802 | 0.027 |
| O(3) | 0.364 | 0.162 | 0.814 | 0.022 |
| O(4) | 0.453 | −0.467 | 0.859 | 0.024 |
| O(5) | 0.448 | −0.290 | 0.793 | 0.023 |
| O(6) | 0.839 | −0.357 | 0.829 | 0.022 |
| O(7) | 0.299 | −0.109 | 0.878 | 0.024 |
| C(1) | 0.544 | −0.105 | 0.865 | 0.018 |
| C(2) | 0.707 | −0.102 | 0.903 | 0.024 |
| C(3) | 0.696 | −0.268 | 0.932 | 0.024 |
| C(4) | 0.510 | −0.303 | 0.958 | 0.031 |
| C(5) | 0.512 | −0.459 | 0.983 | 0.036 |
| C(6) | 0.700 | −0.579 | 0.980 | 0.041 |
| N(7) | 0.884 | −0.548 | 0.955 | 0.037 |
| C(8) | 0.880 | −0.395 | 0.932 | 0.030 |
| O(1W) | 1.051 | −0.114 | 0.732 | 0.030 |
| O(2W) | 1.216 | −0.763 | 0.915 | 0.038 |

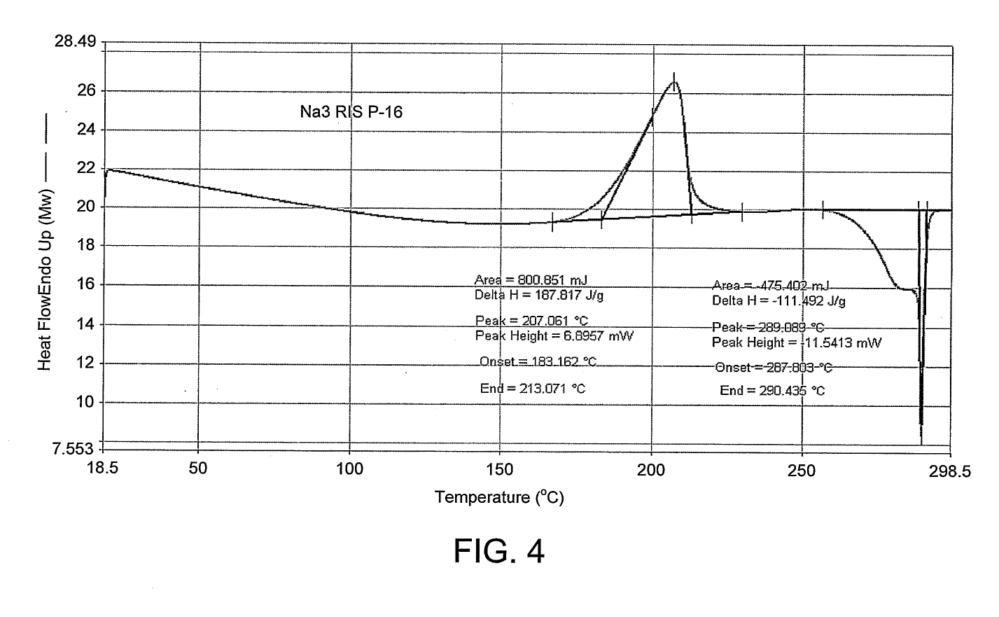

Tri-sodium risedronate dihydrate can be still further characterised by a typical DSC thermograph as shown in FIG. 4. Tri-sodium risedronate dihydrate has a DSC endotherm in the range of 183° C. to 213° C.

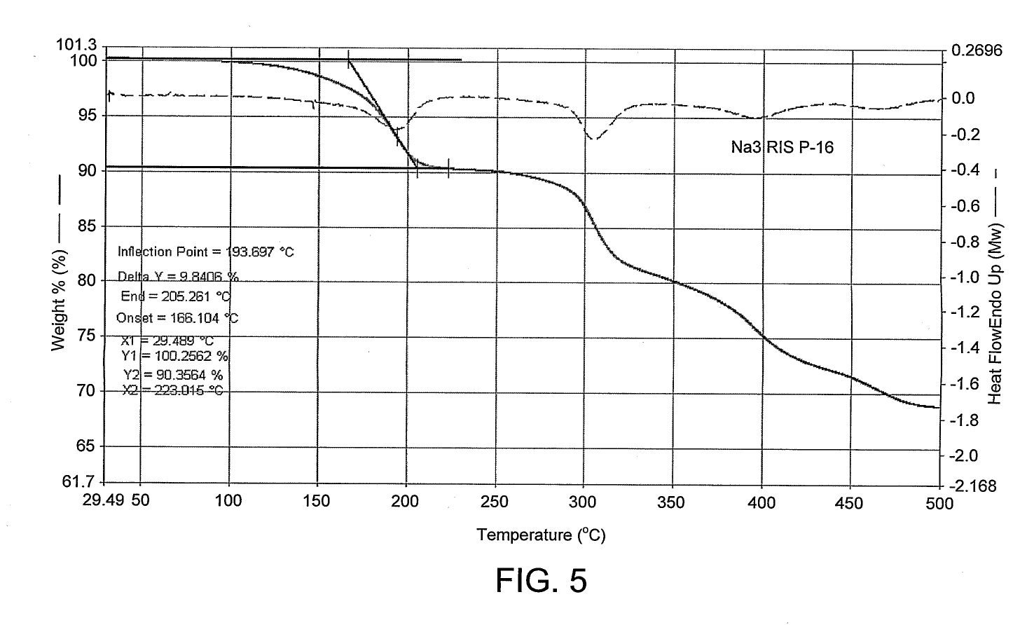

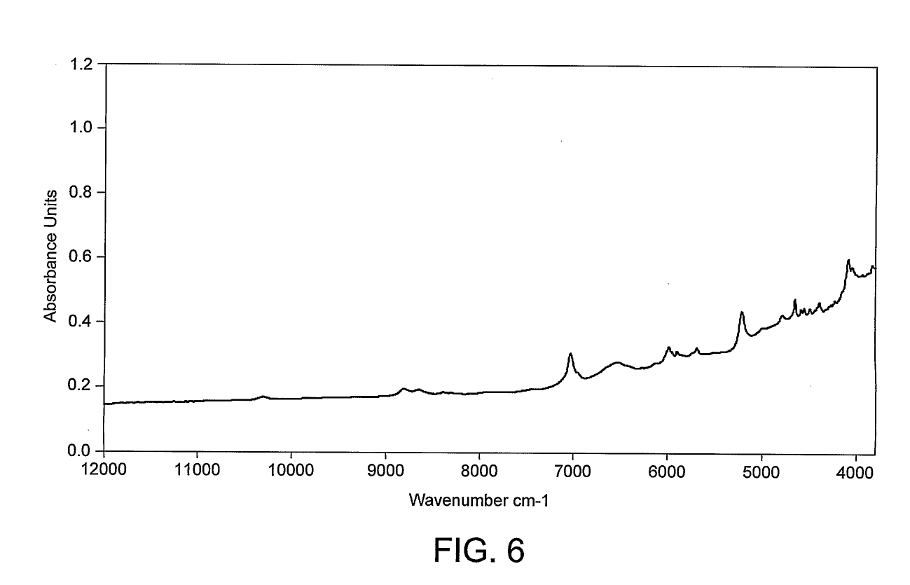

Tri-sodium risedronate as provided by the present invention is further characterised by a TGA weight loss of about 10%, which confirms that tri-sodium risedronate as prepared according to the present invention is present as the dihydrate. This is further illustrated by reference to FIG. 5. Furthermore, a NIR spectrum of tri-sodium risedronate as provided by the present invention is illustrated in FIG. 6, which shows a sharp peak at 5200 cm⁻¹ characteristic of O—H stretching from crystal water.

As used herein, the term “TGA” refers to thermogravimetric analysis. The Karl Fisher assay for determining water content is used which is described in Pharmacopeial Form, Vol 24, No 1, p 5438 (January-February 1998). Such an assay permits the determination of water content of a crystal form based on the Loss on Drying Method. TGA is a measure of the thermally induced weight loss of a material as a function of the applied temperature. TGA is restricted in transitions that involve either a gain or a loss of mass and it is most commonly used to study desolvation processes and compound decomposition.

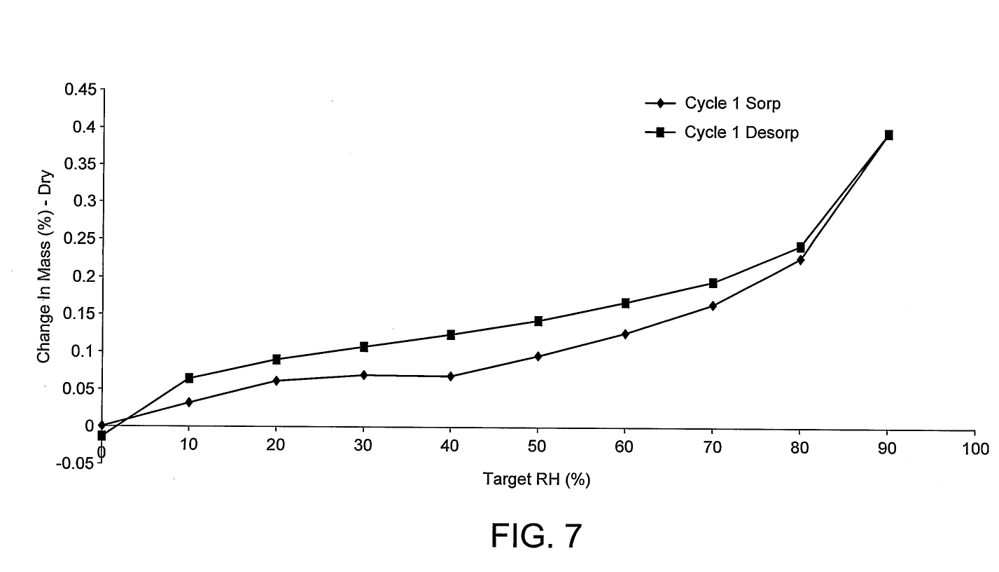

Substantially as hereinbefore described tri-sodium risedronate dihydrate as provided by the present invention is advantageous in view of the non-hygroscopic properties associated with this product and as such the beneficial stability, shell life and formulation properties thereof. The non-hygroscopic nature of tri-sodium risedronate dihydrate as provided by the present invention can be substantiated by reference to FIG. 7, from which it can be seen that when the humidity is raised over a time period of 1200 minutes, the tri-sodium risedronate dihydrate of the invention absorbs a minimum quantity of water.

There is also provided by the present invention a hydrate mixture with comprises (i) a dihydrate form of a tri-(alkali metal) salt of risedronic acid according to the present invention, in particular tri-sodium risedronate dihydrate, together with (ii) a different hydrate form of a salt of risedronic acid formed with the same alkali metal as the dihydrate form. Typically, the ratio of a tri-(alkali metal) salt of risedronic acid according to the present invention to the other hydrate form as present in such a hydrate mixture is about (50-100):(50-0).

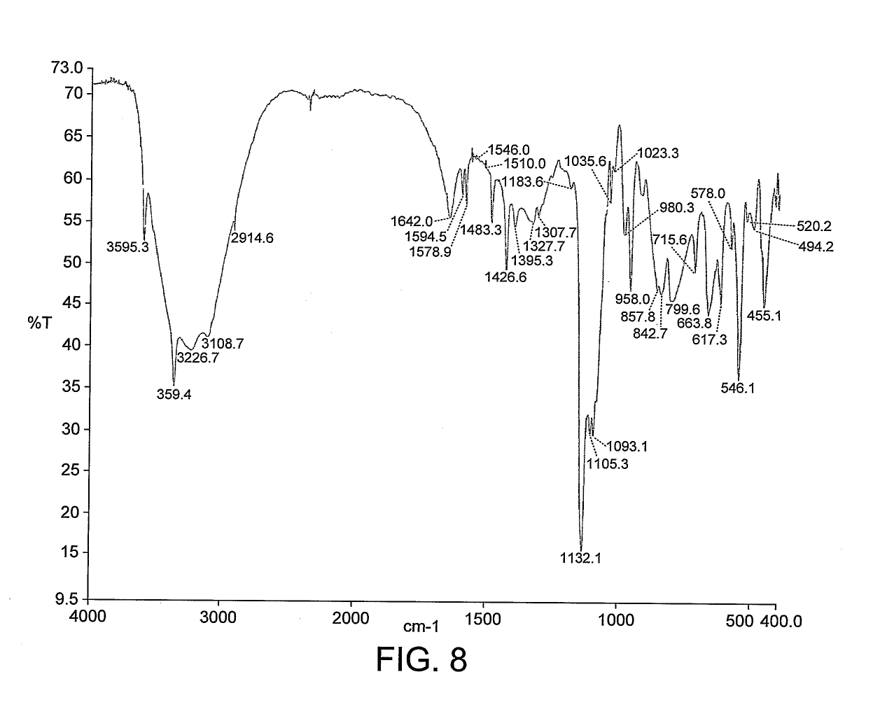

A hydrate mixture as provided by the present invention can be characterised as having an IR pattern, or substantially the same IR pattern, as shown in FIG. 8. More particularly, a hydrate mixture according to the present invention has characteristic IR absorbance at about 3596±4, 3358±4, 3102±4, 1640±4, 1594±4, 1579±4, 1426±4, 1132±4, 1094±4, 958±4 and 545±4 cm⁻¹.

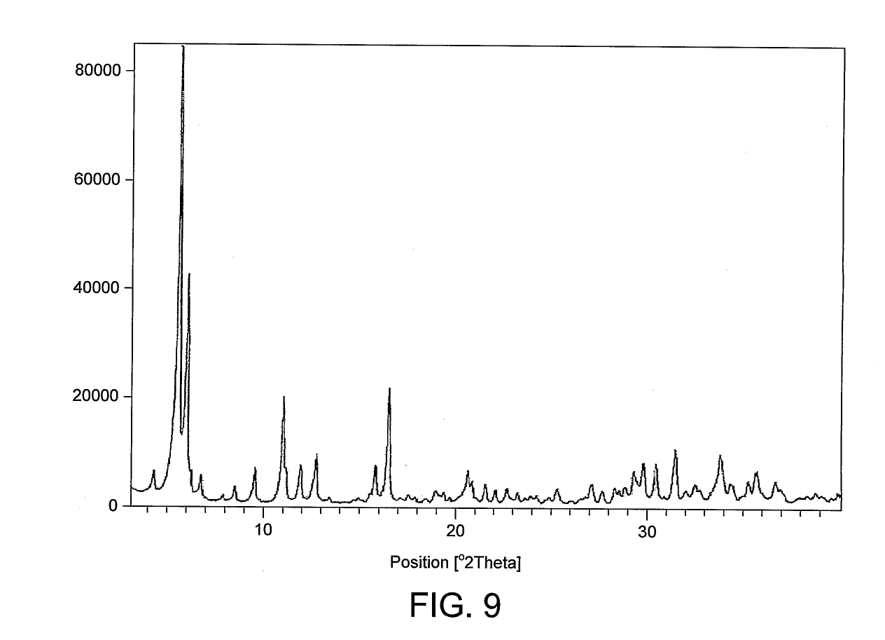

A hydrate mixture as provided by the present invention can be further characterised as having an X-ray powder diffraction pattern, or substantially the same X-ray powder diffraction pattern, as shown in FIG. 9.

A hydrate mixture according to the present invention is further characterised as having characteristic peaks (2θ): 4.3±0.2°, 5.4±0.2°, 6.0±0.2° and 16.5±0.2°. A hydrate mixture according to the present invention is still further characterised by the following other typical peaks (2θ): 9.5±0.2°, 11.0±0.2°, 12.7±0.2°, 15.8±0.2° and 20.6±0.2°.

The present invention also provides a process of preparing a tri-(alkali metal) salt of risedronic acid according to the present invention, or a hydrate mixture, substantially as hereinbefore described, which comprises contacting a suspension of risedronic free acid which a source of a pharmaceutically acceptable alkali metal, adjusting the pH to about 8.5 to 9.5, and thereby converting the risedronic free acid to a tri-(alkali metal) salt of risedronic acid, or a hydrate mixture, according to the present invention substantially as hereinbefore described.

Preferably the source of the pharmaceutically acceptable alkali metal is the corresponding alkali metal hydroxide, preferably sodium hydroxide, whereby addition of the hydroxide achieves adjustment to the above referred to pH range of 8.5 to 9.5. In a preferred embodiment of the present invention, a process as described herein prepares tri-sodium risedronate dihydrate.

Suitably in a process according to the present invention a suspension of risedronic free acid and water is heated to a temperature in the range of about 50° C. to 80° C., preferably in the range of about 60° C. to 70° C., followed by the addition of a hydroxide of the pharmaceutically acceptable alkali metal, in particular sodium hydroxide, to form a solution. Suitably the pH is adjusted to a range of about 8.5 to 9.5 by the addition of the alkali metal hydroxide as described above, and more preferably to a pH in the range of about 9.0 to 9.1. The resulting solution is typically heated to reflux, suitably at about 100° C., and preferably a C₁₋₄alcohol, such as methanol or ethanol, is added. Subsequent cooling results in crystallization of a tri-(alkali metal) salt of risedronic acid, or a hydrate mixture, according to the present invention.

A tri-(alkali metal) salt of risedronic acid, or a hydrate mixture, as provided by the present invention has therapeutic utility in the treatment of diseases associated with bone resorption disorders and more specifically in the treatment of diseases of bone and calcium metabolism. Such diseases include osteoporosis, hyperparathyroidism, hypercalcemia of malignancy, ostolytic bone metastases, myosistis ossifcans progressiva, calcinoisis universalis, arthritis, neuritis, bursitis, tendonitis and other inflammatory conditions.

The present invention further provides, therefore, a pharmaceutical composition comprising a therapeutically effective dose of a tri-(alkali metal) salt of risedronic acid, or a hydrate mixture, substantially as hereinbefore described, together with a pharmaceutically acceptable carrier, diluent or excipient therefor. Excipients are chosen according to the pharmaceutical form and the desired mode of administration.

As used herein, the term “therapeutically effective amount” means an amount of a tri-(alkali metal) salt of risedronic acid, or a hydrate mixture, according to the invention, which is capable of preventing, ameliorating or eliminating a bone resorption disorder.

By “pharmaceutically acceptable” it is meant that the carrier, diluent or excipient is compatible with a tri-(alkali metal) salt of risedronic acid, or a hydrate mixture, according to the invention, and not deleterious to a recipient thereof.

In the pharmaceutical compositions of the present invention for oral, sublingual, subcutaneous, intramuscular, intravenous, topical, intratracheal, intranasal, transdermal or rectal administration, the tri-(alkali metal) salt of risedronic acid is administered to animals and humans in unit forms of administration, mixed with conventional pharmaceutical carriers, for the prophylaxis or treatment of the above disorders or diseases. The appropriate unit forms of administration include forms for oral administration, such as tablets, gelatin capsules, powders, granules and solutions or suspensions to be taken orally, forms for sublingual, buccal, intratracheal or intranasal administration, forms for subcutaneous, intramuscular or intravenous administration and forms for rectal administration. For topical application, a tri-(alkali metal) salt of risedronic acid, or a hydrate mixture, according to the present invention can be used in creams, ointments or lotions.

To achieve the desired prophylactic or therapeutic effect, the dose of a tri-(alkali metal) salt of risedronic acid, or a hydrate mixture, according to the present invention can vary between about 0.01 and about 50 mg per kg of body weight per day. Each unit dose can contain from about 0.1 to about 1000 mg, preferably about 1 to about 500 mg, of a tri-(alkali metal) salt of risedronic acid, or a hydrate mixture, according to the present invention, in combination with a pharmaceutical carrier. This unit dose can be administered 1 to 5 times a day so as to administer a daily dosage of about 0.5 to about 5000 mg, preferably about 1 to about 2500 mg.

When a solid composition in the form of tablets is prepared, the tri-(alkali metal) salt of risedronic acid, or a hydrate mixture, is mixed with a pharmaceutical vehicle such as gelatin, starch, lactose, magnesium stearate, talc, gum arabic or the like. The tablets can be coated with sucrose, a cellulose derivative or other appropriate substances, or else they can be treated so as to have a prolonged or delayed activity and so as to release a predetermined amount of active principle continuously. The use of tablets is generally preferred for a tri-(alkali metal) salt of risedronic acid, or a hydrate mixture, as provided by the present invention.

A preparation in the form of gelatin capsules can be obtained by mixing the tri-(alkali metal) salt of risedronic acid, or a hydrate mixture, according to the present invention, with a diluent and pouring the resulting mixture into soft or hard gelatin capsules.

A preparation in the form of a syrup or elixir or for administration in the form of drops can contain the tri-(alkali metal) salt of risedronic acid, or a hydrate mixture, typically in conjunction with a sweetener, which is preferably calorie-free, optionally antiseptics such as methylparaben and propylparaben, as well as a flavoring and an appropriate color.

Water-dispersible granules or powders can contain the tri-(alkali metal) salt of risedronic acid, or a hydrate mixture, mixed with dispersants or wetting agents, or suspending agents such as polyvinylpyrrolidone, as well as with sweeteners or taste correctors.

Rectal administration is effected using suppositories prepared with binders which melt at the rectal temperature, for example polyethylene glycols.

Parenteral administration is effected using aqueous suspensions, isotonic saline solutions or sterile and injectable solutions which contain pharmacologically compatible dispersants and/or wetting agents, for example propylene glycol or butylene glycol.

A tri-(alkali metal) salt of risedronic acid, or a hydrate mixture, according to the present invention can also be formulated as microcapsules, with one or more carriers or additives if appropriate.

There is also provided by the present invention a tri-(alkali metal) salt of risedronic acid, or a hydrate mixture, substantially as hereinbefore described for use in therapy.

The present invention further provides a tri-(alkali metal) salt of risedronic acid, or a hydrate mixture, substantially as hereinbefore described, for use in the manufacture of a medicament for the treatment of a disease state prevented, ameliorated or eliminated by the administration of an inhibitor of bone resorption. More specifically, the present invention provides a tri-(alkali metal) salt of risedronic acid, or a hydrate mixture, substantially as hereinbefore described, for use in the manufacture of a medicament for the treatment of diseases of bone and calcium metabolism, and even more specifically for the treatment of any one of the following: osteoporosis, hyperparathyroidism, hypercalcemia of malignancy, ostolytic bone metastases, myosistis ossifcans progressiva, calcinoisis universalis, arthritis, neuritis, bursitis, tendonitis and other inflammatory conditions.

The present invention also provides a method of treating a disease state prevented, ameliorated or eliminated by the administration of an inhibitor of bone resorption in a patient in need of such treatment, which method comprises administering to the patient a therapeutically effective amount of a tri-(alkali metal) salt of risedronic acid, or a hydrate mixture, substantially as hereinbefore described. More specifically, the present invention provides a method of treating diseases of bone and calcium metabolism, such as osteoporosis, hyperparathyroidism, hypercalcemia of malignancy, ostolytic bone metastases, myosistis ossifcans progressiva, calcinoisis universalis, arthritis, neuritis, bursitis, tendonitis and other inflammatory conditions, in a patient in need of such treatment, which method comprises administering to the patient a therapeutically effective amount of a tri-(alkali metal) salt of risedronic acid, or a hydrate mixture, substantially as hereinbefore described.

The term “about” as used herein means within an acceptable error range for the particular value as determined by one of ordinary skill in the art, which will depend in part on how the value is measured or determined, i.e., the limitations of the measurement system. For example, at about can mean within 1 or more than 1 standard deviations, per the practice in the art. Alternatively, “about” can mean a range of up to 20%, desirably up to 10%, more desirably up to 5%, and even more desirably up to 1% of a given value. Where particular values are described in the application and claims, unless otherwise stated the term “about” meaning within an acceptable error range for the particular value should be assumed.

The present invention can be further illustrated by the following Figures and non-limiting Examples.

With reference to the Figures, these are as follows:

FIG. 1: An X-ray powder diffraction pattern of tri-sodium risedronate dihydrate according to the present invention obtained by using CuKα radiation on a powder sample collected using a PANalytical X'PertPRO powder diffractometer.

FIG. 2: An IR pattern of tri-sodium risedronate dihydrate obtained by using Perkin Elmer Spectrum GX FT-IR Spectrometer (Detector: DTGS, Beam splitter: extended KBr, Spectral Range: 4000-400 cm⁻¹, Resolution: 4cm⁻¹, 4 scans, Samples prepared as KBr pellets).

FIG. 3: Part of crystal structure of tri-sodium risedronate dihydrate obtained using a Bruker Nonius FR591/Kappa CCD diffractometer with CuKα radiation.

FIG. 4: A DSC pattern of tri-sodium risedronate dihydrate obtained by using a Perkin Elmer DSC Pyris 1, where the sample is scanned at 10° C./min in N₂ atmosphere in closed Al pan.

FIG. 5: A TGA and DTGA thermogram of tri-sodium risedronate dihydrate obtained by using a Perkin Elmer TGA Pyris 7, where the sample is scanned at 10° C./min in N₂ atmosphere in closed Pt pan.

FIG. 6: A NIR spectrum of tri-sodium risedronate dihydrate, obtained by using Bruker NIR Multi Purpose Analyser (MPA). (The spectra were recorded in a diffuse reflectance mode using integrating sphere for collecting reflecting beams. The measurements were carried out over the range 4000 cm⁻¹-12000 cm⁻¹, with a resolution of 8 cm⁻¹. The spectra were averaged over 32 scans. The system was governed via the software OPUS that includes routines for acquisition and processing of spectra). The spectrum shows a sharp peak at 5200 cm⁻¹ characteristic of O—H stretching from crystal water.

FIG. 7: Izotherm diagram for tri-sodium risedronate dihydrate showing the sorption and desorption isotherm for tri-sodium risedronate dihydrate measured in a humidity range from 0-90% RH at 25° C. at DVS 1 (Surface Measurement System).

FIG. 8: An IR pattern of tri-sodium risedronate dihydrate, present in a hydrate mixture with a further hydrate of a sodium risedronate salt, obtained by using Perkin Elmer Spectrum GX FT-IR Spectrometer (Detector: DTGS, Beam splitter: extended KBr, Spectral Range: 4000-400 cm⁻¹, Resolution: 4 cm⁻¹, 4 scans, Samples prepared as KBr pellets).

FIG. 9: An X-ray powder diffraction pattern of tri-sodium risedronate dihydrate, present in a hydrate mixture with a further hydrate form of a sodium risedronate salt, according to the present invention obtained by using CuKα radiation on a powder sample collected using a PANalytical X'PertPRO powder diffractometer.

The following examples are for the purpose of illustration of the invention only and are not intended in any way to limit the scope of the present invention. It will thus be readily apparent to one skilled in the art that varying substitutions and modifications may be made to the invention disclosed herein without departing from the scope and spirit of the invention. Thus, it should be understood that although the present invention has been specifically disclosed by preferred embodiments and optional features, modification and variation of the concepts herein disclosed may be resorted to by those skilled in the art, and that such modifications and variations are considered to be falling within the scope of the invention.

### EXAMPLES

### Example 1

50 ml of water and 15 g of risedronic acid were charged to a 250 ml three necked flask. The suspension was heated to 60° C. and the pH was adjusted with sodium hydroxide (40%) until a pH of about 9 was achieved. The solution was heated to reflux (˜100° C.) and 60 ml of methanol were slowly added under reflux. Crystallization of the salt started at about 78° C., when 50 ml of methanol were added. When all 60 ml of methanol were added, the suspension was maintained at the reflux temperature (˜77° C.) for five minutes, and then allowed to cool. The suspension was then slowly cooled to 0-5° C. over the period of two hours and retained for one hour at this temperature. Risedronate tri-sodium salt, 10.9 g, was obtained after filtration, washed with 20 ml of a water/methanol cold solution (1/1) and dried. Analysis carried out confirmed the risedronate tri-sodium salt thus prepared to be tri-sodium risedronate dihydrate.

### Example 2

100 ml of water and 15 g of risedronic acid were charged to a 500 ml three necked flask. The suspension was heated to 60° C. and the pH was adjusted with sodium hydroxide (40%) until a pH of about 9 was achieved. The solution was heated to reflux (˜100° C.) and 110 ml of methanol were slowly added under reflux. Crystallization of the salt started at about 76° C., when 100 ml of methanol were added. When all 110 ml of methanol were added, the suspension was maintained at the reflux temperature (˜75° C.) for five minutes, and then allowed to cool. The suspension was then slowly cooled to 0-5° C. over the period of two hours and retained for one hour at this temperature. Risedronate tri-sodium salt, 11.7 g, was obtained after filtration, washed with 20 ml of a water/methanol cold solution (1/1) and dried. Analysis carried out confirmed the risedronate tri-sodium salt thus prepared to be tri-sodium risedronate dihydrate as was prepared in Example 1.

### Example 3

50 ml of water and 15 g of risedronic acid were charged to a 500 ml three necked flask. The suspension was heated to 70° C. and the pH was adjusted with sodium hydroxide (40%) until a pH of about 9 was achieved. The solution was heated to reflux (−100° C.) and 200 ml of methanol were slowly added under reflux. Crystallization of the salt started when about 20 ml of methanol were added. When all 200 ml of methanol were added, the suspension was maintained at the reflux temperature (˜77° C.) for five minutes, and then allowed to cool. The suspension was then slowly cooled to 0-5° C. over the period of two hours and retained for one hour at this temperature. Risedronate tri-sodium salt, 17.2 g, was obtained after filtration, washed with 20 ml of a water/methanol cold solution (1/1) and dried. Analysis carried out confirmed the risedronate tri-sodium salt thus prepared to be tri-sodium risedronate dihydrate as prepared in either Example 1 or 2, present in a hydrate mixture with a further hydrate of a sodium risedronate salt.

### Example 4

50 ml of water and 15 g of risedronic acid were charged to a 500 ml three necked flask. The suspension was heated to 60° C. and the pH was adjusted with sodium hydroxide (40%) until a pH of about 9 was achieved. The solution was heated to reflux (˜100° C.) and 200 ml of ethanol were slowly added under reflux. Crystallization of the salt started when about 20 ml of ethanol were added. When all 200 ml of ethanol were added, the suspension was maintained at the reflux temperature (˜78° C.) for five minutes, and then allowed to cool. The suspension was then slowly cooled to 0-5° C. in the period of two hours and retained for one hour at this temperature. Risedronate tri-sodium salt, 19.75 g, was obtained after filtration, washed with 80 ml of ethanol cold solution and dried. Analysis carried out confirmed the risedronate tri-sodium salt thus prepared to be tri-sodium risedronate dihydrate as prepared in either Example 1 or 2, present in a hydrate mixture with a further hydrate of a sodium risedronate salt.

### Example 5

20 ml of water and 15 g of risedronic acid were charged to a 500 ml three necked flask. The suspension was heated to 60° C. and the pH was adjusted with sodium hydroxide (40%) until a pH of about 9 was achieved. The solution was heated to reflux (˜100° C.) and 200 ml of ethanol were slowly added under reflux. Crystallization of the salt started when about 20 ml of ethanol were added. When all 200 ml of ethanol were added, the suspension was maintained at the reflux temperature (˜78° C.) for thirty minutes, and then allowed to cool. The suspension was then slowly cooled to 0-5° C. in the period of two hours and retained for one hour at this temperature. Risedronate tri-sodium salt, 20.19 g, was obtained after filtration, washed with 80 ml of ethanol cold solution and dried. Analysis carried out confirmed the risedronate tri-sodium salt thus prepared to be tri-sodium risedronate dihydrate as prepared in either Example 1 or 2, present in a hydrate mixture with a further hydrate of a sodium risedronate salt.

1. A pharmaceutically acceptable tri-(alkali metal) salt of risedronic acid, which is present as the dihydrate form.

2. A pharmaceutically acceptable tri-(alkali metal) salt of risedronic acid according to claim 1, where the alkali metal is either sodium or potassium.

3. A pharmaceutically acceptable tri-(alkali metal) salt of risedronic acid according to claim 2, where the alkali metal is sodium.

4. A pharmaceutically acceptable tri-(alkali metal) salt of risedronic acid according to claim 2, where the alkali metal is potassium.

5. Tri-sodium risedronate dihydrate.

6. Tri-sodium risedronate dihydrate according to claim 5 having an X-ray powder diffraction pattern, or substantially the same X-ray powder diffraction pattern, as shown in FIG. 1.

7. Tri-sodium risedronate dihydrate according to claim 5 having characteristic X-ray powder diffraction peaks (2θ): 5.4±0.2°, 11.0±0.2° and 16.5±0.2°.

8. Tri-sodium risedronate dihydrate according to claim 7, having the following other typical peaks (2θ): 15.8±0.2°, 20.6±0.2°, 20.8±0.2°, 22.0±0.2°, 25.3±0.2°, 30.4±0.2°, 31.4±0.2° and 33.7±0.2°.

9. Tri-sodium risedronate dihydrate according to claim 5 having an IR pattern, or substantially the same IR pattern, as shown in FIG. 2.

10. Tri-sodium risedronate dihydrate according to claim 5 having characteristic IR absorbance at about 3596±4, 3358±4, 3102±4, 1640±4, 1594±4, 1579±4, 1426±4, 1132±4, 1094±4, 958±4 and 545±4 cm⁻¹.

11. Tri-sodium risedronate dihydrate according to claim 5 having a crystalline structure substantially as shown in FIG. 3.

12. Tri-sodium risedronate dihydrate according to claim 5 having an orthorombic space group P 2₁2₁2₁ and having unit cell parameters comprising crystal axis lengths of a=5.70(2)Å, b=7.25(2)Å, c=32.28(4)Å.

13. Tri-sodium risedronate dihydrate according to claim 5 having a typical DSC thermograph as shown in FIG. 4.

14. Tri-sodium risedronate dihydrate according to claim 5 having a DSC endotherm in the range of 183° C. to 213° C.

15. Tri-sodium risedronate according to claim 5 having a TGA weight loss of about 10%, wherein said tri-sodium risedronate is present as the dihydrate form.

16. A hydrate mixture which comprises (i) a dihydrate form of a tri-(alkali metal) salt of risedronic acid as claimed in claim 1, together with (ii) a different hydrate form of a salt of risedronic acid formed with the same alkali metal as the dihydrate form.

17. A hydrate mixture according to claim 16, wherein the ratio of said tri-(alkali metal) salt of risedronic acid to the other hydrate form is about (50-100):(50-0).

18. A hydrate mixture as claimed in claim 16 characterised as having an IR pattern, or substantially the same IR pattern, as shown in FIG. 8.

19. A hydrate mixture as claimed in claim 16 having characteristic IR absorbance at about 3596±4, 3358±4, 3102±4, 1640±4, 1594±4, 1579±4, 1426±4, 1132±4, 1094±4, 958±4 and 545±4 cm⁻¹.

20. A hydrate mixture as claimed in claim 16 having an X-ray powder diffraction pattern, or substantially the same X-ray powder diffraction pattern, as shown in FIG. 9.

21. A hydrate mixture as claimed in claim 16 having characteristic X-ray powder diffraction peaks (2θ): 4.3±0.2°, 5.4±0.2°, 6.0±0.2° and 16.5±0.2°.

22. A hydrate mixture as claimed in claim 21, having the following other typical peaks (2θ): 9.5±0.2°, 11.0±0.2°, 12.7±0.2°, 15.8±0.2° and 20.6±0.2°.

23. A hydrate mixture as claimed in claim 16, wherein said alkali metal is sodium, as present in both (i) said dihydrate form of said tri-(alkali metal) salt of risedronic acid, and (ii) said different hydrate form of said alkali metal salt of risedronic acid.

24. A process of preparing a tri-(alkali metal) salt of risedronic acid as claimed in claim 1, or a hydrate mixture thereof, or the sodium salt of the hydrate mixture, which comprises
contacting a suspension of risedronic free acid with a source of a pharmaceutically acceptable alkali metal,
adjusting the pH of the suspension to about 8.5 to 9.5, and
thereby converting the risedronic free acid to a tri-(alkali metal) salt of risedronic acid as claimed in claim 1, or a hydrate mixture, or the sodium salt of the hydrate mixture,
wherein the hydrate mixture comprises (i) the dihydrate form of the tri-(alkali metal) salt of risedronic acid as claimed in claim 1, together with (ii) a different hydrate form of a salt of risedronic acid formed with the same alkali metal as the dihydrate form, and
wherein the sodium salt of the hydrate mixture comprises (i) the dihydrate form of the tri-(sodium) salt of risedronic acid, together with (ii) a different hydrate form of the tri-(sodium) salt of risedronic acid.

25. A process as claimed in claim 24, wherein the source of the pharmaceutically acceptable alkali metal is the corresponding alkali metal hydroxide and addition thereof achieves said pH adjustment to a range of about 8.5 to 9.5.

26. A process as claimed in claim 25, wherein said alkali metal hydroxide comprises sodium or potassium hydroxide.

27. A process as claimed in claim 26, wherein said alkali metal hydroxide comprises sodium hydroxide.

28. A process as claimed in claim 27, which prepares tri-sodium risedronate dihydrate.

29. A process as claimed in claim 26, wherein said alkali metal hydroxide comprises potassium hydroxide.

30. A process as claimed in claim 24, wherein said suspension of risedronic free acid and water is heated to a temperature in the range of about 50° C. to 80° C., followed by the addition of said hydroxide of the pharmaceutically acceptable alkali metal to form a solution.

31. A process as claimed in claim 24 wherein said pH is adjusted to a range of about 9.0 to 9.1.

32. A process as claimed in claim 30, wherein said solution is heated to reflux and a C₁₋₄alcohol is added.

33. A process as claimed in claim 32, wherein said C₁₋₄alcohol is methanol or ethanol.

34. A pharmaceutical composition comprising a therapeutically effective dose of a tri-(alkali metal) salt of risedronic acid as claimed in claim 1 or a hydrate mixture thereof, or the sodium salt of the hydrate mixture together with a pharmaceutically acceptable carrier, diluent or excipient therefore, wherein the hydrate mixture comprises (i) the dihydrate form of the tri-(alkali metal) salt of risedronic acid as claimed in claim 1, together with (ii) a different hydrate form of a salt of risedronic acid formed with the same alkali metal as the dihydrate form, and wherein the sodium salt of the hydrate mixture comprises (i) the dihydrate form of the tri-(sodium) salt of risedronic acid, together with (ii) a different hydrate form of the tri-(sodium) salt of risedronic acid.

35-38. (canceled)

39. A method of treating a disease state prevented, ameliorated or eliminated by the administration of an inhibitor of bone resorption in a patient in need of such treatment, which method comprises administering to the patient a therapeutically effective amount of a tri-(alkali metal) salt of risedronic acid as claimed in claim 1, or a hydrate mixture thereof, or a sodium salt of the hydrate mixture, wherein the hydrate mixture comprises (i) the dihydrate form of the tri-(alkali metal) salt of risedronic acid as claimed in claim 1, together with (ii) a different hydrate form of a salt of risedronic acid formed with the same alkali metal as the dihydrate form, and wherein the sodium salt of the hydrate mixture comprises (i) the dihydrate form of the tri-(sodium) salt of risedronic acid, together with (ii) a different hydrate form of the tri-(sodium) salt of risedronic acid.

40. A method of treating diseases of bone and calcium metabolism, in a patient in need of such treatment, which method comprises administering to the patient a therapeutically effective amount of a tri-(alkali metal) salt of risedronic acid as claimed in claim 1, or a hydrate mixture thereof, or a sodium salt of the hydrate mixture, wherein the hydrate mixture comprises (i) the dihydrate form of the tri-(alkali metal) salt of risedronic acid as claimed in claim 1, together with (ii) a different hydrate form of a salt of risedronic acid formed with the same alkali metal as the dihydrate form, and wherein the sodium salt of the hydrate mixture comprises (i) the dihydrate form of the tri-(sodium) salt of risedronic acid, together with (ii) a different hydrate form of the tri-(sodium) salt of risedronic acid.

41. A method of treating any one of the following: osteoporosis, hyperparathyroidism, hypercalcemia of malignancy, ostolytic bone metastases, myosistis ossifcans progressive, calcinoisis universalis, arthritis, neuritis, bursitis, tendonitis and other inflammatory conditions, in a patient in need of such treatment, which method comprises administering to the patient a therapeutically effective amount of a tri-(alkali metal) salt of risedronic acid as claimed in claim 1, or a hydrate mixture thereof, or a sodium salt of the hydrate mixture, wherein the hydrate mixture comprises (i) the dihydrate form of the tri-(alkali metal) salt of risedronic acid as claimed in claim 1, together with (ii) a different hydrate form of a salt of risedronic acid formed with the same alkali metal as the dihydrate form, and wherein the sodium salt of the hydrate mixture comprises (i) the dihydrate form of the tri-(sodium) salt of risedronic acid, together with (ii) a different hydrate form of the tri-(sodium) salt of risedronic acid.
